(19) United States
(12) Patent Application Publication
(10) Pub. No.: US 2016/0305924 A1
(43) Pub. Date: Oct. 20, 2016

(54) METHODS FOR CALIBRATING THE ADMINISTRATION OF VANOXERINE FOR TERMINATING ACUTE EPISODES OF CARDIAC ARRHYTHMIA, RESTORING NORMAL SINUS RHYTHM, PREVENTING RECURRENCE OF CARDIAC ARRHYTHMIA AND MAINTAINING NORMAL SINUS RHYTHM IN MAMMALS

(71) Applicant: LAGUNA PHARMACEUTICALS , INC., La Jolla, CA (US)

(72) Inventor: Arthur M. BROWN, Brecksville, OH (US)

(21) Appl. No.: 14/781,287

(22) PCT Filed: Apr. 25, 2014
(86) PCT No.: PCT/US2014/035553

§ 371 Date: Sep. 29, 2015

Related U.S. Application Data
(60) Provisional application No. 61/816,214, filed on Apr. 26, 2013.
(60) Provisional application No. 61/904,753, filed on Nov. 15, 2013.
(60) Provisional application No. 61/905,054, filed on Nov. 15, 2013.
(60) Provisional application No. 61/915,792, filed on Dec. 13, 2013.

(51) Int. Cl. G01N 33/487; G01N 30/72; A61K 31/495

(52) U.S. Cl. CPC G01N 33/487; A61K 31/495; G01N 30/7266

(57) ABSTRACT

Disclosed embodiments are related to methods for administering compositions of vanoxerine (GBR 12909) to patients by calibrating an effective dose for treatment of cardiac arrhythmias.

### FIELD OF THE INVENTION

Presently disclosed embodiments are related to methods for calibrating the administration of vanoxerine for terminating acute episodes of cardiac arrhythmia, preventing the same, restoring normal sinus rhythm, preventing recurrence of cardiac arrhythmia, and maintaining normal sinus rhythm in mammals. Presently disclosed embodiments particularly relate to methods for dosing and treating mammals with vanoxerine where said mammal is experiencing cardiac arrhythmia or has previously experienced cardiac arrhythmias.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732.) Vanoxerine has also been used for treating and preventing cardiac arrhythmia in mammals. (See U.S. Pat. No. 6,743,797 and U.S. Pat. No. 7,700,600.)

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. In the US alone, AF currently afflicts more than 2.3 million people. By 2050, it is expected that there will be more than 12 million individuals afflicted with AF. While AF is rarely a lethal arrhythmia, it is responsible for substantial morbidity and can lead to complications such as the development of congestive heart failure or thromboembolism. Currently available Class I and Class III anti-arrhythmic drugs reduce the rate of recurrence of AF, but are of limited use because of a variety of potentially adverse effects, including ventricular proarrhythmia. Because current therapy is inadequate and fraught with side effects, there is a clear need to develop new therapeutic approaches.

Current first line pharmacological therapy options for AF include drugs for rate control. Despite results from several studies suggesting that rate control is equivalent to rhythm control, many clinicians believe that patients are likely to have better functional status when in sinus rhythm. Further, being in AF may introduce long-term mortality risk, where achievement of rhythm control may improve mortality.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, these agents have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Prior studies have been performed using single dose administration of flecainide or propafenone (Class I drugs) in terminating atrial fibrillation. Particular studies investigated the ability to provide patients with a known dose of one of the two drugs so as to self-medicate should cardiac arrhythmia occur. P. Alboni, et al., “Outpatient Treatment of Recent-Onset Atrial Fibrillation with the ‘Pill-in-the-Pocket’ Approach,” NEJM 351; 23 (2004); L. Zhou, et al., “‘A Pill in the Pocket’ Approach for Recent Onset Atrial Fibrillation in a Selected Patient Group,” Proceedings of UCLA Healthcare 15 (2011). However, the use of flecainide and propafenone has been criticized as including candidates having structural heart disease and thus providing patients likely to have risk factors for stroke who should have received antithrombotic therapy, instead of the flecainide or propafenone. NEJM 352:11 (Letters to the Editor) (Mar. 17, 2005). Similarly, the use of warfarin concomitantly with propafenone was criticized.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A Third Class of Anti-Arrhythmic Action: Effects on Atrial and Ventricular Intracellular Potentials and other Pharmacological Actions on Cardiac Muscle of MJ 1999 and AH 3747,” (Br. J. Pharmacol 39:675-689 (1970), and Singh B. N., Vaughan Williams E. M., “The Effect of Amiodarone, a New Anti-Anginal Drug, on Cardiac Muscle,” Br. J. Pharmacol 39:657-667 (1970)), but these are not selective Class III agents. Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)). Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (I_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{K}, (rapidly activating and deactivating) and I_{Ks} (slowly activating and deactivating). (Sanguinetti and Jurkiewicz, “Two Components of Cardiac Delayed Rectifier K+Current. Differential Sensitivity to Block by Class III Anti-Arrhythmic Agents,” J Gen Physiol 96:195-215 (1990)). I_{K}, is also the product of the human ether-a-go-go gene (hERG). Expression of hERG cDNA in cell lines leads to production of the hERG current which is almost identical to I_{K}, (Curran et al., “A Molecular Basis for Cardiac Arrhythmia: hERG Mutations Cause Long QT Syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2, 4′-piperidin]-6yl], (+)-, monochloride (MK-499) predominantly, if not exclusively, block I_{Kr}. Although amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, as a nonspecific adrenergic blocker, acts as an inhibitor of the enzyme phospholipase, and causes pulmonary fibrosis (Nademanee, K., “The Amiodarone Odessey.” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness, by prolonging APD, prevents and/or terminates reentrant arrhythmias. Most selective Class III antiarrhythmic agents currently in development, such as d-sotalol and dofetilide predominantly, if not exclusively, block I_{Kr}, the rapidly activating component of I_{K} found both in atria and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M., “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supraventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced Effectiveness and Dangers of Reverse use Dependence,” Circulation, 81:686-690 (1990); Sadanaga et al., “Clinical Evaluation of the Use-Dependent QRS Prolongation and the Reverse Use-Dependent QT Prolongation of Class III Anti-Arrhythmic Agents and Their Value in Predicting Efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-Dependent Prolongation of Cardiac Action Potentials by a Methanesulfonanilide Class III Anti-Arrhythmic Agent: Specific Block of Rapidly Activating Delayed Rectifier K+ current by Dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

Vanoxerine has been indicated for treatment of cardiac arrhythmias. Indeed, certain studies have looked at the safety profile of vanoxerine and stated that no side-effects should be expected with a daily repetitive dose of 50 mg of vanoxerine. (U. Sogaard, et. al., “A Tolerance Study of Single and Multiple Dosing of the Selective Dopamine Uptake Inhibitor GBR 12909 in Healthy Subjects,” International Clinical Psychopharmacology, 5:237-251 (1990)). However, Sogaard, et. al. also found that upon administration of higher doses of vanoxerine, some effects were seen with regard to concentration difficulties, increase systolic blood pressure, asthenia, and a feeling of drug influence, among other effects. Sogaard, et. al. also recognized that there were unexpected fluctuations in serum concentrations with regard to these healthy patients. While they did not determine the reasoning, control of such fluctuations may be important to treatment of patients.

Further studies have looked at the ability of food to lower the first-pass metabolism of lipophilic basic drugs, such as vanoxerine. (S. H. Ingwersen, et. al., “Food Intake Increases the Relative Oral Bioavailability of Vanoxerine,” Br. J. Clin. Pharmac; 35:308-130 (1993)). However, no methods have been utilized or identified for treatment of cardiac arrhythmias in conjunction with the modulating effects of food intake.

It is desirable to optimize methods for administering vanoxerine to patients exhibiting signs of, or experiencing episodes of cardiac arrhythmia in view of the problems associated with current anti-arrhythmic agents. Indeed, there remains a need for a method for the effective treatment of cardiac arrhythmias in mammals with vanoxerine, where the methods comprise steps to calibrate a dose with regard to the particular patient and the particular patients profile, and/or to calibrate a dose based on the profile of an individual patient to provide for safe and effective treatment of cardiac arrhythmia and restoration of normal sinus rhythm through administration of effective amounts of vanoxerine.

### SUMMARY

Embodiments of the present disclosure relate to methods for calibrating the dosage for vanoxerine administration comprising: administering a first dose of vanoxerine; measuring the physiological concentration of vanoxerine, modifying the dosage of vanoxerine based on the physiological concentration after the first administration; and administering at least a second modified dosage of vanoxerine.

An additional aspect of the present disclosure relates to a method for determining a treatment dosage of vanoxerine for treatment of cardiac arrhythmia in a patient who has already received a first dose of vanoxerine; wherein a physiological level of vanoxerine is evaluated from said first administered dosage of vanoxerine, and prescribing for the patient, a subsequent treatment of vanoxerine for treating cardiac arrhythmia based on the evaluated physiological level.

An additional aspect of the present disclosure is a method for treating a second or later episode of cardiac arrhythmia, wherein the patient was previously treated for a first episode of cardiac arrhythmia with a first dosage of vanoxerine, the method comprising administering a second dosage of vanoxerine to the patient upon the occurrence of a subsequent episode of cardiac arrhythmia, wherein the amount of the second dosage of vanoxerine is based on a measure of a pharmacokinetic response of the patient to the first dose of vanoxerine.

An additional aspect of the present disclosure includes a method for treating cardiac arrhythmias in a mammal comprising: administering a first dosage of vanoxerine to a mammal; measuring the pharmacokinetic response to said first dosage administration; administering at least a second dosage wherein said at least second dosage is calibrated according to the pharmacokinetic response from the first dosage administration.

Other aspects of the present disclosure include methods for treating a patient exhibiting symptoms of cardiac arrhythmia, comprising: administering an initial dosage of vanoxerine in a first course of treatment, thereby treating the cardiac arrhythmia; measuring the pharmacokinetic value of vanoxerine or a metabolite thereof for the patient resulting from the first course of treatment; determining a desirable vanoxerine dosage for the patient based on the measured pharmacokinetic value; and administering the desirable vanoxerine dosage to the patient in a subsequent course of treatment to treat the same or a subsequent episode of cardiac arrhythmia.

Other aspects of the present disclosure include methods of achieving and/or maintaining a pre-determined plasma level comprising administering an initial dosage of vanoxerine in a first course of treatment; measuring the plasma levels of vanoxerine; comparing the measured plasma levels to the pre-determined plasma level; modifying a subsequent dose of vanoxerine to meet the pre-determined plasma level; and administering the modified dose of vanoxerine in a subsequent course of treatment to treat the same or a subsequent episode of cardiac arrhythmia.

Other aspects of the present disclosure include methods for terminating acute episodes of cardiac arrhythmia, such as atrial fibrillation or ventricular fibrillation, in a mammal, such as a human, by administering to that mammal at least an effective amount of vanoxerine to terminate an acute episode of cardiac arrhythmia; measuring a pharmacokinetic response to that effective amount of vanoxerine; determining a modified dose of vanoxerine based on the measured pharmacokinetic response, and administering the modified dose of vanoxerine to said patient.

Another aspect of the present disclosure includes a method for restoring normal sinus rhythm in a mammal, such as a human, exhibiting cardiac arrhythmia by administering at least a first effective dose of vanoxerine to restore normal sinus rhythm; measuring a pharmacokinetic response to that effective amount of vanoxerine; determining a modified dose of vanoxerine based on the measured pharmacokinetic response, and administering the modified dose of vanoxerine to said patient.

Another aspect of the present disclosure includes a method for maintaining normal sinus rhythm in a mammal, such as a human, by administering at least a first effective dose of vanoxerine to restore normal sinus rhythm; measuring a pharmacokinetic response to that effective amount of vanoxerine; determining a modified dose of vanoxerine based on the measured pharmacokinetic response, and administering the modified dose of vanoxerine to said patient.

Another aspect of the present disclosure includes a method for preventing a recurrence of an episode of cardiac arrhythmia in a mammal, such as a human, by administering at least a first effective dose of vanoxerine to restore normal sinus rhythm; measuring a pharmacokinetic response to that effective amount of vanoxerine; determining a modified dose of vanoxerine based on the measured pharmacokinetic response, and administering the modified dose of vanoxerine to said patient.

Further embodiments include methods for treating cardiac arrhythmia in a mammal, such as a human, by administering a first effective dose of vanoxerine to treat the cardiac arrhythmia; measuring a pharmacokinetic response to that effective amount of vanoxerine; determining a modified dose of vanoxerine based on the measured pharmacokinetic response, and administering the modified dose of vanoxerine to said patient.

Other methods of the disclosure include methods for modulation of Cₘₐₓ (maximum peak concentration) and tₘₐₓ (the time after administration when the drug reaches maximum plasma concentration) with regard to a particular patient, wherein a first effective dose of a drug comprising vanoxerine is administered; the pharmacokinetic response to the administered vanoxerine is measured subsequent to administration; the Cₘₐₓ and tₘₐₓ are determined for the patient; and further effective doses of vanoxerine are determined so as to modulate one or more of the Cₘₐₓ and tₘₐₓ of the patient.

A method for calibrating the dosage for vanoxerine administration to a patient comprising: administering a first dose of vanoxerine to a patient; measuring the patient's physiological concentration of vanoxerine; modifying the dosage of vanoxerine based on the measured physiological concentration after the first administration; and administering the modified dosage of vanoxerine to the patient.

A method for calibrating a dosage of vanoxerine to a pre-determined plasma level, comprising: administering an initial dosage of vanoxerine in a first course of treatment; measuring the plasma level of vanoxerine for the patient resulting from the first course of treatment at a pre-determined time; comparing the measured plasma level of vanoxerine to the pre-determine plasma level; determining a desirable vanoxerine dosage for the patient based on a comparison between the measured plasma level and the pre-determined plasma level; and administering the desirable vanoxerine dosage to the patient in a subsequent course of treatment to treat a subsequent episode of cardiac arrhythmia.

A method for treating cardiac arrhythmias in a mammal comprising: administering a first dosage of vanoxerine to a mammal; measuring the pharmacokinetic response to said first dosage administration; administering at least a second dosage wherein said at least second dosage is calibrated according to the pharmacokinetic response from the first dosage administration.

Administering steps in any of the foregoing methods may comprise administration by a caregiver, a medical professional, or self-administered by a patient.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

As used herein, “calibrate” means modification of the unit dose of vanoxerine, whether up or down, to change the given dose.

As used herein, “administering” or “administer” refers to the actions of a medical professional or caregiver, or alternatively self-administration by the patient.

The term “steady state” means wherein the overall intake of a drug is fairly in dynamic equilibrium with its elimination.

As used herein, a “pre-determined” plasma level or other physiological tissue or fluid and refers to a concentration of vanoxerine at a given time point. Typically, a pre-determined level will be compared to a measured level, and the time point for the measured level will be the same as the time point for the pre-determined level. In considering a pre-determined level with regard to steady state concentrations, or those taken over a period of hours, the pre-determined level is referring to the mean concentration taken from the area under the curve (AUC), as the drug increases and decreases in concentration in the body with regard to the addition of a drug pursuant to intake and the elimination of the drug via bodily mechanisms.

Cardiac arrhythmias include atrial, junctional, and ventricular arrhythmias, heart blocks, sudden arrhythmic death syndrome, and include bradycardias, tachycardias, re-entrant, and fibrillations. These conditions, including the following specific conditions: atrial flutter, atrial fibrillation, multifocal atrial tachycardia, premature atrial contractions, wandering atrial pacemaker, supraventricular tachycardia, AV nodal reentrant tachycardia, junctional rhythm, junctional tachycardia, premature junctional contraction, premature ventricular contractions, ventricular bigeminy, accelerated idioventricular rhythm, monomorphic ventricular tachycardia, polymorphic ventricular tachycardria, and ventricular fibrillation, and combinations thereof are all capable of severe morbidity and death if left untreated. Methods and compositions described herein are suitable for the treatment of these and other cardiac arrhythmias.

Vanoxerine, is susceptible to metabolism by numerous mechanisms, including CYP3A4 among other known P450 cytochromes. Certain advantages exist by eliminating first pass metabolism by enzymatic catalysts and increasing the bioavailability with regard to a given dose of vanoxerine. Accordingly, the bioavailability of a given dose of vanoxerine can be modified by blocking and/or having a CYP3A4 or other P450 antagonist so as to prevent the metabolism of vanoxerine before it has the ability to act on its intended target. This provides for the opportunity to modify the dose of vanoxerine needed to provide for suitable levels of vanoxerine in the body for treatment of the cardiac arrhythmias.

Interestingly, certain foods, whether high in fat or low in fat diet also impact metabolism and bioavailability for some or all patients. (S. H. Ingwersen, et. al., “Food Intake Increases the Relative Oral Bioavailability of Vanoxerine,” Br. J. Clin. Pharmac; 35:308-310 (1993)). As patients typically continue a normal diet while taking medications, the input of particular foods or dosing strategies that accompany such diet is evaluated. Accordingly, despite the variability within patients and within the patient population as a whole, it is necessary and advantageous to provide methods for administration of vanoxerine for treatment of cardiac arrhythmias. In particular, these methods are utilized to improve efficacy, improve safety, and increase the efficiency of vanoxerine for treatment of cardiac arrhythmias and for conversion of patients to normal sinus rhythm.

Accordingly, in view of the known variability with regarding to vanoxerine, it is advantageous to provide methods that calibrate a given dose with regard to a particular patient. In one embodiment, calibrating provides that a first dose of vanoxerine is administered to a patient. After the administration, the resulting plasma levels (or other fluid or tissues of the patient) are then measured to determine levels of vanoxerine in the body of the patient. Based on the measured levels, further doses of vanoxerine can be increased or decreased to provide for efficacious and safe administration. In a further embodiment, the measured levels are then reviewed and compared to known pharmacological profiles to determine how to proceed with treatment. In some cases, the measured pharmacological levels are compared to a goal plasma level for the particular patient at a particular time point, whereby the concentration of vanoxerine is increased via administration of additional vanoxerine, or is allowed to decrease naturally within the patient. Accordingly, based on the measured pharmacological levels and the resulting effects, subsequent suitable doses can be determined for the patient to achieve desired levels should a further episode of cardiac arrhythmia occur.

Treatment of patient's exhibiting cardiac arrhythmia may therefore be improved by measuring and calibrating the amounts of vanoxerine in the blood. This is particularly suited, as human patients have shown variability within the Cₘₐₓ concentration levels subsequent to administration of vanoxerine, therefore, some calibration and modification of dosing can improve treatment. Accordingly, while a given dose may typically be sufficient for most patients, other patients may require additional dosing or larger doses based on individual response. Such individualized and calibrated treatment provides greater efficacy for patients, increases safety profile, and provides increased ability to treat cardiac arrhythmia.

Methods of administering vanoxerine to patients based on their metabolism profile can be utilized to improve the efficacy of vanoxerine in the treatment of cardiac arrhythmias. For example, in the case of a fast metabolism, vanoxerine is metabolized by first pass metabolism and thereby limiting the effective Cₘₐₓ. Modulation of the Cₘₐₓ then comprises a method of administering a first dosage of vanoxerine, measuring the concentration of vanoxerine in the patient in tissue, blood, plasma, or other fluids at a given time point subsequent to the administration of the first dose, adjusting the dosage of vanoxerine to increase or decrease the Cₘₐₓ based on the results of the measured concentrations; and administering a further dose of vanoxerine at the adjusted dosage rate provides for improved therapeutic levels for the patient.

Where a first administered dose is sufficient to meet a pre-determine plasma level, or has otherwise converted the patient to normal sinus rhythm, the patient may be monitored to ensure that the normal sinus rhythm is maintained. However, where the concentration of vanoxerine is higher than necessary for conversion, a future dose may be modified to a lower amount of vanoxerine administered to the patient. Where the patient has not converted to normal sinus rhythm, or the given dose is not reaching pre-determined plasma levels, administration of additional vanoxerine may be administered. In some cases this administration is given via a bolus dose, or in an IV line, or in other cases as an injection, sublingual, buccal, orally, or via other suitable mechanism for administration to the patient.

Vanoxerine, once in the body, metabolizes, at least partially, into at least five different metabolites, including: M01, M02, M03, M04, and M05. Accordingly, it is advantageous to modify and calibrate not only the measured amounts of vanoxerine, but in some embodiments also to measure and calibrate the metabolites thereof. Accordingly, a method comprises administering a first dose of vanoxerine, measuring the concentrations of vanoxerine and one or more of metabolites M01, M02, M03, M04, and M05 at a time subsequent to the administration of the first dose of vanoxerine, and modifying a further dose to be administered to the patient based on the measured concentration. Accordingly, subsequent treatment may be modified based on the results so as to provide either a single course treatment, or profile that a treatment plan may require two or more doses to provide for efficacious treatment.

Because of metabolic variability of vanoxerine and its metabolites, but also with regard to Cₘₐₓ and tₘₐₓ, the ability to modify and calibrate treatment allows for a targeted approach to treating an individual and also allows for safe and efficacious dosing of patients should they undergo additional episodes of cardiac arrhythmia. Accordingly, after a first dose, second and subsequent doses may be modified to increase or decrease the dosage to modify the concentration to a preferred or optimal concentration for treatment of the cardiac arrhythmia. Accordingly, as needed, the patient is given a subsequent dose of vanoxerine that is particularly tailored to their individual metabolism and pharmacokinetic response to vanoxerine.

In some embodiments, the goal Cₘₐₓ plasma concentration may be 5 ng/ml to 1000 ng/ml at one hour post administration. Indeed, for treatment of an acute episode of cardiac arrhythmia concentrations of about 10 ng/ml to about 400 ng/ml at one hour post administration are preferred. For preventative administration to prevent episodes of cardiac arrhythmia, concentrations of about 10 ng/ml to about 400 ng/ml at one hour post administration are preferred. For maintenance of normal sinus rhythm, concentrations of about 10 ng/ml to about 400 ng/ml at one hour post administration are preferred. For restoring normal sinus rhythm, concentrations of about 10 ng/ml to about 400 ng/ml at one hour post administration are preferred. For preventing recurrence of cardiac arrhythmia, concentrations of about 10 ng/ml to about 400 ng/ml at one hour post administration are preferred where each are preferred in the range of about 20 to about 200 ng/ml.

Pharmaceutical concentrations in the body are often measured from plasma. However, other suitable measurements may be taken from other bodily fluids, and determined by appropriate means. The Cₘₐₓ plasma concentrations of for effective treatment of cardiac arrhythmia are typically between about 20 and about 400 ng/ml. However, in some cases the Cₘₐₓ is between about 10 and about 500 ng/ml. In preferred embodiments, the Cₘₐₓ plasma concentration is between about 20 and about 200 ng/ml, or about 20 and about 150 ng/ml, or about 25 and about 125 ng/ml, and in some cases between about 40 and about 100 ng/ml. At a Cₘₐₓ plasma concentration of more than 40 ng/ml, the majority of patients convert to normal sinus rhythm in under about 12 hours, and at 60 ng/ml the rate of conversion is higher and faster. Accordingly, in some embodiments, a plasma concentration of at least 60 ng/ml is preferred.

In certain circumstances, it is important to maintain a plasma concentration of vanoxerine or one or more metabolites for a given time period. For example, it is advantageous to maintain a plasma level within the range of about 5 to 400 ng/ml of vanoxerine for a period of about 1 to about 24 hours to restore normal sinus rhythm, or arrest an episode of cardiac arrhythmia. Doing so may require the administration of multiple doses over a given time period. Accordingly, methods of administration provide for the ability to calibrate dosing for effective treatment of cardiac arrhythmias. Wherein the first does is administered and the pharmacokinetic response is measured in the patient, and subsequent doses are modified based on the measured response so as to provide for efficacious plasma levels. Alternative embodiments utilize an elevated physiological level for more than a day. Indeed, it may be advantageous to provide for elevated physiological level for days, weeks, months, and/or years to maintain sinus rhythm and to prevent recurrence of cardiac arrhythmia. Accordingly a daily or multiple times a day dose may be appropriate in some circumstances for providing such elevated levels.

In some embodiments, e.g. for the treatment of adult humans, a dosage of 1 mg to 1000 mg per unit dose is appropriate to reach a Cₘₐₓ, tₘₐₓ, AUC, or other pre-determined measurement. Other embodiments may utilize a dosage of about 50 mg to 800 mg, or about 25 to 100 mg, or about 100 mg to about 600 mg, or about 200 to about 400 mg. Other preferred doses are 25, 50, 75, 100, 150, 200, 300, 400, and 500 mg of vanoxerine.

Plasma level concentrations (and other physiological concentrations) are modified by the methods described herein. Wherein the physiological concentration may be taken from other body tissues that can measure amounts of vanoxerine in the body. Because of variability with regarding to first pass metabolism of vanoxerine the step of measuring the plasma level concentration at a given time point is advantageous to tailor a dose to an individual. Preferred plasma level concentrations, taken at a time point of 1 hour post administration are about 5 to about 1000 ng/ml. In alternative embodiments, plasma level concentrations at 1 hour post administration are about 10 to about 400 ng/ml, or about 20 to about 200 ng/ml, or about 25 to about 150 ng/ml or about 40 to about 100 ng/ml, and about 60 to about 100 ng/ml. Alternatively, plasma level concentrations may be taken at a time point of about 90 minutes, 2 hours, 3 hours, 4 hours, 6 hours, 8 hours, 12 hours, and 24 hours post administration, or taken a multiple time points to provide additional data for maximizing the calibration and modification of doses in the methods described herein.

Accordingly, certain methods may comprise administration of a given dose of vanoxerine; measuring plasma levels at two or more time points; modifying the unit dose of vanoxerine in view of the plasma levels; administration of a second dose of vanoxerine based on the adjusted unit dose; measuring plasma levels at one or more additional time points; modifying the unit dose a second time to further calibrate the dose for the given patient; and administering a third, or subsequent dose of vanoxerine. The ability to dose, measure, and modify subsequent doses provides for greater precision with regard to the targeted plasma levels for efficacious treatment while limiting side effects. Measurement of the other suitable pharmacological levels instead of plasma is suitable in other embodiments.

Additionally, modification of Cₘₐₓ and tₘₐₓ is appropriate to maintain consistent plasma level concentrations. Cₘₐₓ levels are about 5 to about 400 ng/ml. Conversely tₘₐₓ is appropriately reached at about 1-3 hours post administration. In other embodiments, tₘₐₓ is appropriately reached at about 30 minutes, 90 minutes, 120 minutes, 180 minutes, 240 minutes, or about 300 minutes post administration.

A further embodiment utilizes the fact that patients typically split into two groups, fast and slow metabolizers. Accordingly, the method comprises administering vanoxerine, measuring the pharmacokinetic response in the patient, comparing the measured pharmacokinetic response to historical data of fast or slow metabolizers and thereby determining whether the patient is a fast or slow metabolizer. Accordingly, a dose can then be normalized based on whether the patient is a fast or slow metabolizer. This step of determining the pharmacokinetic profile can be utilized with any of the embodiments described herein.

Suitable methods for treatment of cardiac arrhythmias include various dosing schedules which may be administered by any technique capable of introducing a pharmaceutically active agent to the desired site of action, including, but not limited to, buccal, sublingual, nasal, oral, topical, rectal and parenteral administration. Dosing may include single daily doses, multiple daily doses, single bolus doses, slow infusion injectables lasting more than one day, extended release doses, IV or continuous dosing through implants or controlled release mechanisms, and combinations thereof. These dosing regimens in accordance with the method allow for the administration of the vanoxerine in an appropriate amount to provide an efficacious level of the compound in the blood stream or in other target tissues. Delivery of the compound may also be through the use of controlled release formulations in subcutaneous implants or transdermal patches.

For oral administration, a suitable composition containing vanoxerine may be prepared in the form of tablets, dragees, capsules, syrups, and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of vanoxerine. Such tablets may, if desired, be provided with enteric coatings by known method, for example by the use of cellulose acetate phthalate. Suitable binding or granulating agents are e.g. gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing vanoxerine and at least one diluent, and optionally a part of the disintegrating agent, is granulated together with an aqueous, ethanolic or aqueous-ethanolic solution of the binding agents in an appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, vanoxerine and the desired additives may be filled into a capsule, such as a hard or soft gelatin capsule. The contents of a capsule and/or caplet may also be formulated using known methods to give sustained release of the active compound.

Liquid oral dosage forms of vanoxerine may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing vanoxerine may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of vanoxerine may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspension may be prepared from sterile powders or granules having one or more carriers or diluents mentioned for use in the formulations for oral administration. Parenteral administration may be through intravenous, intradermal, intramuscular or subcutaneous injections.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

28 patients participated in a study of vanoxerine. 25 patients took a 300 mg dose of vanoxerine and 3 patients took a placebo. Each patient gave samples before administration of their dose, and then again at nine further time points, 30 minutes after administration, 1, 2, 3, 4, 6, 8, 12, and 24 hours post administration.

Evaluation of samples: A total of 270 human plasma samples were received (in duplicate). The samples were shipped frozen over dry ice. The samples were stored at −70° C. (nominal) until analysis.

Determination of vanoxerine and 17-hydroxyl vanoxerine concentrations in human plasma samples was performed against their respective calibration curve within concentration range of 1 to 250 ng/mL. The content determination of 16-hydroxyl vanoxerine and all other hydroxyl metabolites in human plasma samples was performed against 17-hydroxyl vanoxerine calibration curve within concentration range of 1 to 250 ng/mL.

Frozen samples were thawed at room temperature. Samples, calibrators, QC samples, sample blanks (blank plasma) and reagent blanks (water) were processed at room temperature. To 200 μL aliquots of each sample, calibrator, and QC sample are added 20 μL of 500 ng/mL of internal standard solution. A sample blank (blank plasma) and reagent blank (water) were also prepared, but without addition of internal standard solution (20 μL of diluent was added instead). To each sample, 200 μL of 1% ammonium hydroxide solution were added and vortex mixed. 3.0 mL of methyl tert-butyl ether were added and then vortex mix for 30 seconds. The sample is then centrifuged for 5 minutes at 4000 rpm and then transferred for about 30 minutes at −70° C. (nominal). The upper (organic) layer was transferred into an evaporation tubes and then evaporated under N₂ stream at −50° C. for about 20 min. Samples were then reconstituted in 1000 μL reconstitution solution (80:20:0.1 water: acetonitrile: formic acid). Samples were mixed, allow to stand for about 3 minutes and mixed again. 900 μL of the sample solution was transferred to a 1.5 mL microcentrifuge tube and centrifuged for 5 minutes at 14000 rpm at room temperature. 800 μL of the sample solution was transferred to a glass autosampler vial, and then transferred to the autosampler for analysis. Samples were separated using reversed-phase liquid chromatography with a C18 column.

Chromatographic conditions were as follows:
HPLC instrument: Waters Alliance e2795 HT with temperature controlled autosampler
Column: Waters XBridge C18 3.5μ 100×2.1 mm P.N. 186003022, Lot No. 0143302711 with an appropriate guard column
Column temperature: 45° C.
Autosampler temp. 5° C.
Flow: 0.3 mL/min
Mobile phase A: 10 mM ammonium formate buffer:MeOH:ACN 80:10:10
Mobile phase B: 10 mM ammonium formate buffer:MeOH:ACN 5:50:45
Purge solvent: Water:ACN:formic acid 50:50:0.1
Wash solvent: Water:ACN:formic acid 50:50:0.1
Injection volume: 20 μL
Run Time: 15 minutes

Gradient Table:

| Time (min) | % A | % B | Curve |
|---|---|---|---|
| 0.00 | 100 | 0 | 1 |
| 0.20 | 100 | 0 | 6 |
| 2.00 | 50 | 50 | 6 |
| 6.00 | 50 | 50 | 6 |
| 6.10 | 0 | 100 | 6 |
| 10.90 | 0 | 100 | 6 |
| 11.00 | 100 | 0 | 6 |

Detection was based on electro-spray interface in positive mode (ESI+) LC-MS/MS technique, using Micromass Quattro Premier XE MS/MS detector with MassLynx and QuanLynx software version 4.1. MRM transitions for vanoxerine, 17-hydroxyl vanoxerine (M01) and for internal standard were m/z 451→203, 467→203 and 433→185 respectively. The MRM transition for all hydroxyl metabolites was m/z 467→203.

Calibration curve standards and QC samples were prepared by spiking vanoxerine, 17-hydroxyl vanoxerine and 16-hydroxyl vanoxerine into blank human plasma (with K2EDTA as anticoagulant).

Calibration standards nominal concentrations of 1, 2, 10, 50, 100, 200 and 250 ng/mL and QC samples nominal concentrations of 3, 20, 125 and 187.5 were prepared for each analyte.

The identification of the metabolites was as follows:

| | Retention time | Relative retention time |
|---|---|---|
| Compound | (min) | (RRT) |
| Vanoxerine | 9.21 | 1.00 |
| M03 | 7.38 | 0.81 |
| M04 | 7.88 | 0.86 |
| M01 (17-hydroxyl | 8.51 | 0.92 |
| vanoxerine) | | |
| M02 (16-hydroxyl | 8.74 | 0.95 |
| vanoxerine) | | |
| M05 | 8.96 | 0.97 |

TABLE 1
Concentrations ng/ml

| Time | | | | | | | Total |
|---|---|---|---|---|---|---|---|
| (h) | Vanoxerine | M03 | M04 | M01 | M02 | M05 | Metabolites |
| −15 | 1.00* | 1.00 | 1.00 | 1.00 | 1.00 | 1.00 | 1.00 |
| .5 | 25.26 | 1.02 | 10.79 | 1.93 | 1.00 | 1.30 | 12.44 |
| 1 | 70.09 | 2.46 | 49.74 | 7.51 | 1.02 | 1.88 | 60.41 |
| 2 | 104.98 | 7.08 | 82.62 | 19.65 | 1.02 | 2.59 | 111.20 |
| 3 | 81.43 | 7.21 | 75.63 | 18.68 | 1.01 | 2.14 | 102.83 |
| 4 | 54.30 | 7.54 | 63.85 | 16.42 | 1.01 | 1.45 | 88.35 |
| 6 | 32.85 | 6.59 | 48.14 | 11.48 | 1.00 | 1.22 | 66.35 |
| 8 | 24.37 | 4.92 | 38.38 | 8.98 | 1.00 | 1.21 | 52.45 |
| 12 | 15.89 | 3.98 | 26.84 | 6.30 | 1.00 | 1.05 | 37.05 |
| 24 | 8.29 | 2.32 | 13.46 | 3.66 | 1.00 | 1.01 | 19.07 |

*A quantity of (1.00) represents an amount that was below the lower limit of quantitation, which is <1.139 ng/ml vanoxerine, and <1.1141 ng/ml 17-hydroxyl vanoxerine.

TABLE 2
Standard Deviations

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 43.77 | 0.12 | 15.58 | 3.20 | 0.00 | 0.80 | 19.28 |
| 1 | 61.82 | 2.51 | 49.96 | 7.08 | 0.10 | 1.13 | 59.70 |
| 2 | 100.18 | 4.70 | 51.64 | 15.31 | 0.07 | 2.56 | 70.07 |
| 3 | 80.40 | 5.40 | 49.04 | 13.63 | 0.07 | 2.31 | 64.45 |
| 4 | 55.01 | 5.32 | 39.75 | 11.31 | 0.04 | 1.16 | 52.50 |
| 6 | 35.74 | 5.10 | 31.30 | 7.90 | 0.00 | 0.87 | 41.84 |
| 8 | 30.37 | 4.05 | 25.29 | 6.74 | 0.00 | 0.94 | 33.41 |
| 12 | 24.03 | 3.15 | 17.62 | 4.70 | 0.00 | 0.27 | 23.17 |
| 24 | 10.34 | 2.11 | 8.91 | 2.76 | 0.00 | 0.03 | 12.31 |

Table 2 shows the standard deviations from the above 25 patients receiving vanoxerine. The three patients receiving a placebo are not included in the data and all data points indicated levels of vanoxerine below the lower limit of quantitation.

Tables 1 and 2, above, show tests of 25 patients with a 300 mg dose of vanoxerine. Blood was drawn from each of the test patients before the administration of the vanoxerine, and then at 9 additional time points, one half hour after administration, then 1, 2, 3, 4, 6, 8, 12, and 24 hours subsequent to administration.

The 25 patients fall into two categories: 15 fell into a category of having the majority of time point levels that were below the average mean (as identified in Table 1) “low concentration group average,” and the remaining 10 patients had the majority of time points above the average mean “high concentration group average.”

TABLE 3
Low concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 16.99 | 1.00 | 12.17 | 1.52 | 1.00 | 1.37 | 13.39 |
| 1 | 40.07 | 2.78 | 56.35 | 6.76 | 1.03 | 1.73 | 66.46 |
| 2 | 42.50 | 6.48 | 74.06 | 14.09 | 1.00 | 1.30 | 94.80 |
| 3 | 31.40 | 5.36 | 59.58 | 11.38 | 1.00 | 1.14 | 76.25 |
| 4 | 24.40 | 5.91 | 51.98 | 10.34 | 1.00 | 1.05 | 68.14 |
| 6 | 16.69 | 4.96 | 38.61 | 7.08 | 1.00 | 1.00 | 50.52 |
| 8 | 11.82 | 3.29 | 29.92 | 5.30 | 1.00 | 1.00 | 38.45 |
| 12 | 6.31 | 2.58 | 20.60 | 3.67 | 1.00 | 1.00 | 26.71 |
| 24 | 5.01 | 1.79 | 12.09 | 2.66 | 1.00 | 1.00 | 16.08 |

TABLE 4
Low concentration standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 0.00 | 0.00 | 0.00 | 0.00 | 0.00 | 0.00 | 0.00 |
| 1 | 24.47 | 0.00 | 17.68 | 1.67 | 0.00 | 0.98 | 20.45 |
| 2 | 27.50 | 3.10 | 59.32 | 7.56 | 0.13 | 1.05 | 71.04 |
| 3 | 28.16 | 4.18 | 44.96 | 9.05 | 0.00 | 0.58 | 57.77 |
| 4 | 22.66 | 3.28 | 34.95 | 7.06 | 0.00 | 0.46 | 45.53 |
| 6 | 16.11 | 3.72 | 30.77 | 7.28 | 0.00 | 0.16 | 42.04 |
| 8 | 14.20 | 3.51 | 21.42 | 3.71 | 0.00 | 0.00 | 28.30 |
| 12 | 11.19 | 2.27 | 15.60 | 2.86 | 0.00 | 0.00 | 20.34 |
| 24 | 3.07 | 1.69 | 10.44 | 1.72 | 0.00 | 0.00 | 13.40 |

TABLE 5
High concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 37.67 | 1.06 | 8.71 | 2.55 | 1.00 | 1.19 | 11.01 |
| 1 | 115.12 | 1.98 | 39.82 | 8.64 | 1.00 | 2.10 | 51.33 |
| 2 | 198.71 | 7.96 | 95.46 | 28.00 | 1.05 | 4.51 | 135.79 |
| 3 | 156.49 | 9.98 | 99.70 | 29.64 | 1.03 | 3.64 | 142.69 |
| 4 | 96.14 | 9.83 | 80.45 | 24.93 | 1.02 | 2.01 | 116.64 |
| 6 | 57.08 | 9.03 | 62.44 | 18.08 | 1.00 | 1.55 | 90.10 |
| 8 | 43.18 | 7.37 | 51.08 | 14.50 | 1.00 | 1.52 | 73.46 |
| 12 | 29.30 | 5.93 | 35.57 | 9.98 | 1.00 | 1.13 | 51.52 |
| 24 | 3.07 | 1.69 | 10.44 | 1.72 | 0.00 | 0.00 | 13.40 |

TABLE 6
High concentration group standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 62.39 | 0.19 | 12.37 | 4.71 | 0.00 | 0.45 | 18.34 |
| 1 | 72.52 | 1.19 | 31.62 | 6.52 | 0.00 | 1.26 | 38.76 |
| 2 | 96.23 | 5.50 | 60.49 | 19.21 | 0.11 | 3.17 | 82.34 |
| 3 | 77.51 | 6.85 | 58.66 | 13.99 | 0.11 | 3.12 | 70.07 |
| 4 | 63.43 | 6.50 | 46.33 | 10.60 | 0.06 | 1.67 | 54.47 |
| 6 | 44.79 | 6.26 | 38.98 | 8.02 | 0.00 | 1.35 | 48.76 |
| 8 | 40.12 | 4.97 | 32.08 | 7.21 | 0.00 | 1.48 | 38.93 |
| 12 | 33.45 | 3.74 | 22.14 | 5.13 | 0.00 | 0.42 | 26.71 |
| 24 | 14.82 | 3.02 | 11.03 | 3.24 | 0.00 | 0.05 | 14.70 |

As can be seen, in Tables 3 and 5, the low concentration group barely has plasma levels rise above 40 ng/ml at any time point in reference to vanoxerine. Whereas, the high concentration group has levels that rise to nearly 200 ng/ml at a time of two (2) hours after administration. Furthermore, the variability with regard to each of the groups is also wider in the high concentration group average. The standard deviations in Table 4 are lower than those in Table 6, (no T-test or 95% confidence was run), demonstrating that the variability was greater in the high concentration group than the low concentration group.

Accordingly, in view of the data, certain methods may be suitable for normalizing or minimizing the variability with regard to a single dosage of vanoxerine or one or more of the metabolites thereof.

### Example 2

12 subjects received daily doses of Vanoxerine for 11 consecutive days, at doses of 25, 50, 75, and 100 mg, with a 14 day washout period between dose levels.

At 25 mg, plasma levels were not detectable after 8 hours. At 50, 75, and 100 mg doses, plasma levels were detectable at 24 hours and steady state was reached by day 8. PK was linear and dose proportional across 50, 75 and 100 mg doses. The 100 mg QD Cₘₐₓₛₛ and AUC₀₋₂₄ₛₛ suggests a trend toward non-linear PK that may become apparent at doses >100 mg QD. PK was highly variable at steady state; Cₘₐₓ, ss, and AUC₀₋₂₄ₛₛ inter-subject variability ranged from 55-85%. The results are listed below in Table 7.

TABLE 7

| | PK Data | PK Data |
|---|---|---|
| Dose | (Mean +/− SD) C_{Max} | (Mean +/− SD) T_{1/2} |
| 50 mg | 27.5+/21.3 ng/ml | 49.39 +/− 26.18 hr |
| T_{Max} 1.27 +− 0.5 hr | | (4.71-110.57) |
| (0.5-2.0) | | |
| 75 mg | 27.4 +/− 15.5 ng/ml | 52.53 +/− 37.46 |
| | | (10.26-116.67) |
| 100 mg | 40.2 +/− 26.6 ng/ml | 15.38 +/− 43.55 |
| | | (5.56-125.00) |

Data from these studies demonstrates an increased half-life of the drug when daily doses are given. Furthermore, it was noted that heart rate and systolic blood pressure increased slightly in most subjects at 75 and 100 mg doses and did not completely return to baseline during washout between dose levels.

### Example 3

Fourteen healthy patients were given vanoxerine of 25, 75, and 125 mg, daily, for 14 days with a washout of 14 days between dose levels. A standardized meal was served 15 minutes prior to each dosing.

No significant adverse events were seen in any of the studies. Steady-state serum levels were reported within 9-11 days with disproportionately and statistically greater levels at higher doses as compared with the lower doses. The non-linear kinetics may be due to increasing bioavailability at higher doses based on a saturation of first pass metabolism.

### Example 4

Four patients were given 50, 100, and 150 mg vanoxerine, daily, for 7 days.

Upon administration of 100 mg for 7 days, increases in systolic blood pressure and heart rate were seen. Similarly, during the 150 mg test, the patients also saw increases in systolic blood pressure and in heart rate. Steady state levels were achieved within one week for all patients.

### Example 5

3 different cohorts, each including 35 subjects were enrolled in a study with 25 taking vanoxerine and 10 receiving placebo. Cohort 1 included 200 mg vanoxerine, Cohort 2 include 200 or 300 mg of vanoxerine, and Cohort 3 included 200, 300, or 400 mg vanoxerine. The vanoxerine or identical appearing placebo was randomly assigned and administered in a double-blinded fashion.

TABLE 8
Atrial Fibrillation/Flutter history:

| | Placebo (32) | 200 mg (22) | 300 mg (25) | 400 mg (25) |
|---|---|---|---|---|
| A Flutter at | 4 (12.5) | 4 (18.2) | 4 (16) | 4 (16 |
| Entry N (%) | | | | |

Duration of Concurrent AF/AFL Episode

| | | | | |
|---|---|---|---|---|
| Mean, days | 1.84 | 2.33 | 2.43 | 1.97 |
| range, days | 0-6 | 0-6 | 0-6 | 0-7 |
| Rx same day as | 41 | 23 | 32 | 32 |
| onset, % | | | | |

Time since AF/AFL Dx

| | | | | |
|---|---|---|---|---|
| Mean, yrs | 3.9 | 4.8 | 4.5 | 5.1 |
| range, yrs | 0-21 | 0-13 | 0-13 | 1-13 |
| Rx prior DC | 44 | 45 | 52 | 32 |
| cadioversion % | | | | |

Time since last DC Cardioversion

| | | | | |
|---|---|---|---|---|
| Mean, mo | 13.6 | 15.2 | 18.2 | 21 |
| range, mo | 0-77 | 0-5 | 0-90 | 0-103 |

TABLE 9
Efficacy: Percent conversion through 4, 8, and 24 hours

| | Placebo (32) | 200 mg (22) | 300 mg (25) | 400 mg (25) |
|---|---|---|---|---|
| 0-4 hr | 13% | 18% | 40% | 52% |
| 0-8 hr | 23% | 45% | 52% | 76% |
| 0-24 hr | 38% | 59% | 64% | 84% |

Indeed, there is a significant improvement in conversion as compared to placebo at all time-points, wherein the rate of conversion or percent conversion at 0-4 hours, 0-8 hours and 0-24 hours was improved with any dose of vanoxerine. Accordingly, a measurement of the improvement comprises a comparison to the rate of conversion of placebo, wherein the improvement is based on the percent increase in conversion over placebo. The 200 mg, having an improvement of conversion of 38%, 96%, and 55% at the above time points, 300 mg: 207%, 126%, and 68%, and the 400 mg: 300%, 230%, and 121%.

TABLE 10
Time to conversion

| Log-rank test results for time conversion | P-value |
|---|---|
| Overall | 0.0005 |
| Pairwise: 200 mg versus control | 0.0838 |
| pairwise: 300 mg versus control | 0.0180 |
| pairwise: 400 mg versus control | <0.0001 |

Indeed, the time to conversion based on the P-value and the above chart provides that placebo does not have greater than a 40% conversion at any time point below 24 hours, whereas all doses of vanoxerine are greater than 40% conversion at about 7 hours, and conversion greater than 50% for all dose at 12 hours, and nearing 60% at about 16 hours.

TABLE 11
Conversion of Atrial Flutter

| | Placebo (32) | 200 mg (22) | 300 mg (25) | 400 mg (25) |
|---|---|---|---|---|
| A flutter, N | 4 | 4 | 4 | 4 |
| Conversion, % | 25% | 50% | 75% | 75% |

Definition of “pure” atrial flutter: only Atrial Flutter (no AF) seen at −30, −15, and 0 time points. Conversion at any time within 24 hours. No 1:1 AFL seen post dose in any subject.

TABLE 12
Adverse events:

| Placebo (32) | 200 mg (22) | 300 mg (25) | 400 mg (25) |
|---|---|---|---|
| 7 (22%) | 4 (18%) | 7 (28%) | 10 (40%) |
| subjects | subjects | subjects | subjects |
| reporting | reporting | reporting | reporting |
| 10 AE's | 8 AEs (1 SAE) | 12 AEs | 23 AEs (1 SAE) |

In view of doses of 200, 300 and 400 mg, there was a highly statistically significant dose dependent increase in the conversion to sinus rhythm of recent onset symptomatic AF/AFL. The highest oral dose of 400 mg achieved a conversion rate of 76% at 8 hours and 84% within 24 hours. Time to conversion curves also demonstrate increasing slope of conversion with successively higher doses, suggesting a Cₘₐₓ dependent effect.

Vanoxerine was well tolerated at all doses with only two serious adverse events, one at the 200 mg dose and one at the 400 mg dose (the 200 mg dose being an upper respiratory infection, the 400 mg dose being lower extremity edema secondary to amlodipine), neither related to the study drug. Similar to efficacy, there was a dose dependent increase in adverse events, but only the high dose event rate was notably higher than that of the placebo group. Accordingly, vanoxerine has a high degree of efficacy for the conversion of recent onset symptomatic atrial fibrillation and atrial flutter in the absence of proarrhythmia, wherein the conversion rate approaches that of DC cardioversion.

Accordingly, hemodynamic effects on heart rate and systolic blood pressure have been seen with multiple dosing of vanoxerine. Several subjects exhibited dose-related increases in heart rate and systolic blood pressure. These effects, however, do not correlate with vanoxerine concentration AUC and interpretation is further confounded by the lack of placebo-control. These effects do not immediately dissipate upon discontinuation of study drug. It is suggested that vanoxerine exerts an effect on the autonomic nervous system over the course of the study. The lack of correlation with plasma vanoxerine AUC, may be interpreted as either evidence of a significant pharmacodynamic lag in the hemodynamic effects of vanoxerine or evidence that a metabolite is responsible for the hemodynamic effects.

Accordingly, it may be advantageous to determine the profile of the patient because of the known variability with vanoxerine such that any subsequent administration of vanoxerine is determined by the pharmacokinetic profile of the individual patient. A first administration can then be measured and the PK profile of the individual can be determined. A subsequent dose, therefore, can be increased or decreased to meet a particular Cₘₐₓ or other pre-determined physiological concentration. Thereby, the patient takes the minimum vanoxerine needed to establish therapeutic levels to prevent recurrence of cardiac arrhythmia and to maintain normal sinus rhythm based on the particular half-life of the patient.

In other embodiments, a patient may be administered vanoxerine, their pharmacokinetic profile is determined to be either a slow or a fast metabolic profile, and a subsequent dose is determined by administering pre-determined doses based on the known slow or fast metabolic profile of historical patient profiles.

Modulation of a dose provides for greater accuracy with regard to target plasma concentrations for the treatment of cardiac arrhythmia. Utilization of certain methods allows for appropriate modulation of AUC, Cₘₐₓ and tₘₐₓ such that variability is minimized with patients. Wherein, the population displays a high level of variability, but typically falling within a fast and a slow metabolic groups, an understanding of the particular pharmacokinetic profile of the individual patient provides advantages in treatment with vanoxerine. In some embodiment, it is therefore important to understand the pharmacokinetic profile of the individual. Therefore, a determination of whether the individual falls within the fast or slow profile based on predetermined profiles is utilized in a method described herein. Then based on the determined profile, an appropriate dose may be administered. Therefore, the methods provided for herein, provide for greater accuracy with regard to target physiological levels, thus increasing the safety profile, improving efficacy of treatment, and minimizing side effects that may be associated with treatment.

Indeed, whether someone falls within a fast or slow group can be established based on administering a known quantity of vanoxerine and testing the patient at a time point subsequent to administration. This time point may be 30 min, 60 min, 90 min, 120 min, or at 3 or 4 hours post administration. However, based on the known profile of patients, the profile of the individual can be characterized based on the AUC or their Cₘₐₓ or tₘₐₓ at these time points. This determination can then be utilized to modify or calibrate a further dose for the particular treatment or for treatment in a subsequent administration or occurrence of cardiac arrhythmias.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

1. A method for calibrating the dosage for vanoxerine administration to a patient comprising:
a. administering a first dose of vanoxerine to a patient;
b. measuring the patient's physiological concentration of vanoxerine;
c. modifying the dosage of vanoxerine based on the measured physiological concentration after the first administration; and
d. administering the modified dosage of vanoxerine to the patient.

2. The method of claim 1 wherein the physiological concentration is measured from the plasma.

3. The method of claim 1 wherein the physiological level concentration of vanoxerine and at least one metabolite is measured.

4. The method of claim 1 further comprising the step of comparing the measured physiological concentration to a pre-determined physiological level.

5. The method of claim 1 wherein the measured physiological concentration is determined from blood samples drawn from the patient about 1 hour post administration of the first dose of vanoxerine.

6. The method of claim 4 wherein the pre-determined physiological level concentration is determined from blood samples drawn from the patient about 2 hours post administration of the first dose of vanoxerine.

7. The method of claim 4 wherein the pre-determined physiological level concentration is about 10 to about 200 ng/ml at one hour post administration of the first dose of vanoxerine.

8. The method of claim 7 wherein the pre-determined physiological level concentration is about 25 to about 150 ng/ml at one hour post administration of the first dose of vanoxerine.

9. The method of claim 1 further comprising the step of determining whether the patient is a fast or slow metabolizer of vanoxerine, and wherein the modified dosage is based on a known metabolic response based on the fast or slow metabolic status of the patient.

10. The method of claim 3 wherein the metabolite measured is selected from the group consisting of M01, M02, M03, M04, M05, and combinations thereof; and wherein the modification of the dose of vanoxerine is based on the plasma level concentration after the first administration of vanoxerine and the M04 metabolite.

11. A method for treating cardiac arrhythmias in a mammal comprising:
a. administering a first dosage of vanoxerine to a mammal;
b. measuring the pharmacokinetic response to said first dosage administration;
c. administering at least a second dosage wherein said at least second dosage is calibrated according to the pharmacokinetic response from the first dosage administration.

12. The method of claim 11 wherein the pharmacokinetic response is measured as the plasma level concentration of vanoxerine.

13. The method of claim 11, wherein the measured pharmacokinetic response is compared to a pre-determined plasma level concentration.

14. The method of claim 11 wherein the plasma level concentration of vanoxerine and at least one metabolite is measured.

15. The method of claim 13 wherein the pre-determined plasma level concentration is determined from blood samples drawn from the patient about 1 hour post administration of the first dose of vanoxerine.

16. The method of claim 13 wherein the pre-determined plasma level concentration is determined from blood samples drawn from the patient about 2 hours post administration of the first dose of vanoxerine.

17. The method of claim 13 wherein the pre-determined plasma level concentration is about 10 to about 200 ng/ml at one hour post administration of the first dose of vanoxerine.

18. The method of claim 17 wherein the pre-determined plasma level concentration is about 25 to about 150 ng/ml at one hour post administration of the first dose of vanoxerine.

19. A method for treating a patient exhibiting symptoms of cardiac arrhythmia, comprising:
a. administering an initial dosage of vanoxerine in a first course of treatment;
b. measuring a pharmacokinetic value of vanoxerine and/or a metabolite thereof for the patient resulting from the first course of treatment;
c. determining a desirable vanoxerine dosage for the patient base on the measured pharmokinetic value; and
d. administering the desirable vanoxerine dosage to the patient in a subsequent course of treatment to treat a subsequent episode of cardiac arrhythmia.

20. The method of claim 19 wherein the pharmacokinetic value is determined from blood samples drawn from the patient about 1 hour post administration of the first dose of vanoxerine.

21-28. (canceled)
